(19) United States
(12) Patent Application Publication
(10) Pub. No.: US 2022/0096673 A1
(43) Pub. Date: Mar. 31, 2022

(54) TOWEL DISINFECTING AND DRYING MACHINE

(71) Applicant: SHENZHEN ANTOP TECHNOLOGY CO., LTD, SHENZHEN, GD (CN)

(72) Inventors: Ruidian YANG, Shenzhen (CN); Weixin NI, Shenzhen (CN)

(73) Assignee: SHENZHEN ANTOP TECHNOLOGY CO., LTD, Shenzhen, GD (CN)

(21) Appl. No.: 17/134,674

(22) Filed: Dec. 28, 2020

(30) Foreign Application Priority Data
Sep. 25, 2020 (CN) 202022149134.3
Sep. 25, 2020 (CN) 202022150339.3
Oct. 16, 2020 (CN) 202022317590.4

(51) Int. Cl. A61L 2/10; A61L 2/26; D06F 58/20; D06F 58/26

(52) U.S. Cl. CPC A61L 2/10; A61L 2/26; D06F 58/203; D06F 2103/66; A61L 2202/11; A61L 2202/122; A61L 2202/26; D06F 58/26

(57) ABSTRACT

Disclosed is a towel disinfecting and drying machine, including a main machine which includes casing, ultraviolet LED module, heater, and fan. The bottom of the casing is provided with air outlet corresponding to the outlet of the fan and air inlet, and the heater is located in the air flow path of the fan. The ultraviolet LED module includes basal plate installed on the bottom of the casing and ultraviolet LED lamp bead(s) installed on the basal plate. Fan is used to blow out heat for drying; ultraviolet LED module is installed on the bottom of the casing to emit ultraviolet light for disinfecting, which can simplify the structure, is more energy-saving and has a long life; because of the small size of the ultraviolet LED module, easy layout, and small occupied space, the main machine can be made smaller, which is convenient for users to install and use.

### CROSS-REFERENCE TO RELATED APPLICATIONS

This application claims the priority of a Chinese patent application filed at the Chinese Patent Office on Sep. 25, 2020 with the application number 202022149134.3 and titled “Towel disinfecting and drying machine”, the entire content of which is incorporated hereby by reference.

This application claims the priority of a Chinese patent application filed at the Chinese Patent Office on Sep. 25, 2020 with the application number 202022150339.3 and titled “towel disinfector”, the entire content of which is incorporated hereby by reference.

This application claims the priority of a Chinese patent application filed at the Chinese Patent Office on Oct. 16, 2020 with the application number 202022317590.4 and titled “Towel disinfecting and drying machine”, the entire content of which is incorporated hereby by reference.

### TECHNICAL FIELD

The application pertains to the field of towel disinfecting equipment, and more particularly, a towel disinfecting and drying machine.

### BACKGROUND

The statements here only provide background information related to this application, and do not necessarily constitute prior art. Towel disinfecting and drying machines are devices generally used to disinfect and dry towels, fabrics and other fabrics. In the current towel disinfecting and drying machine, an ultraviolet lamp and a heater are generally installed in the main machine to irradiate and heat the towel on the hanger for disinfecting and drying. However, this kind of towel disinfecting and drying machine is highly energy-consuming.

### SUMMARY

The purpose of the embodiments of the present application is to provide a towel disinfecting and drying machine to solve the problem of relatively large energy consumption of the towel disinfecting and drying machine in the related art.

In order to achieve the above purpose, the technical solution used in the embodiments of the present application is to provide a towel disinfecting and drying machine, including a main machine, the main machine including a casing, an ultraviolet LED module installed on the bottom of the casing, and a heater installed in the casing and a fan installed in the casing; an air outlet is arranged on the bottom of the casing corresponding to the outlet of the fan, and the heater is arranged in the air flow path of the fan, an air inlet is arranged on the bottom of the casing, and the ultraviolet LED module includes a basal plate installed on the bottom of the casing and ultraviolet LED lamp bead(s) installed on the basal plate.

The beneficial effects of the towel disinfecting and drying machine provided by the embodiments of the present application are: compared with the prior art, the towel disinfecting and drying machine of the present application has a fan and a heater installed in the casing of the main machine, and the heat created by the heater is blown out by the fan for drying; an ultraviolet LED module is installed on the bottom of the casing to emit ultraviolet light by ultraviolet LED lamp beads for disinfecting, which can simplify the structure of the main machine, and is energy-efficient and has a long service life; because of the small size of the ultraviolet LED module, easy layout, and small occupied space, the main machine can be made smaller, which is convenient for users to install and use.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

In order to more clearly describe the technical solutions in the embodiments of the present application, the following will briefly introduce the accompanying drawings used in the description of the embodiments or exemplary technologies. Obviously, the accompanying drawings in the following description are only some embodiments of the present application. Those skilled in the art can obtain other drawings based on these drawings without creative work.

FIG. 1 is a structural schematic diagram 1 of the towel disinfecting and drying machine provided in Embodiment 1 of the application;

FIG. 2 is a structural schematic diagram 2 of the towel disinfecting and drying machine provided in Embodiment 1 of the application;

FIG. 3 is a schematic cross-sectional structure diagram of the towel disinfecting and drying machine provided in Embodiment 1 of the application;

FIG. 4 is a structural schematic diagram when the air guiding window on the main machine in FIG. 3 is separated;

FIG. 5 is an enlarged view of part A in FIG. 4;

FIG. 6 is a schematic diagram of the exploded structure of the main machine in FIG. 4;

FIG. 7 is an enlarged view of part B in FIG. 6;

FIG. 8 is a structural schematic diagram of the main machine of the towel disinfecting and drying machine provided in Embodiment 2 of the application;

FIG. 9 is a schematic cross-sectional structure diagram of the main machine of the towel disinfecting and drying machine provided in Embodiment 2 of the application;

FIG. 10 is a schematic cross-sectional structure diagram of the towel disinfecting and drying machine provided in Embodiment 3 of the application;

FIG. 11 is a schematic diagram of the exploded structure of the main machine in FIG. 10;

FIG. 12 is a structural schematic diagram of the towel disinfecting and drying machine provided in Embodiment 4 of the application;

FIG. 13 is a schematic diagram of the exploded structure of the towel disinfecting and drying machine provided in Embodiment 4 of the application;

FIG. 14 is a schematic cross-sectional structure diagram of the main machine in FIG. 13;

FIG. 15 is the first schematic diagram of the exploded structure of the main machine in FIG. 13;

FIG. 16 is the second schematic diagram of the exploded structure of the main machine in FIG. 13;

FIG. 17 is a structural schematic diagram of the turntable in FIG. 16;

FIG. 18 is a structural schematic diagram when the air guiding window on the main machine provided by Embodiment 5 of the application is separated;

FIG. 19 is a structural schematic diagram of a transmitting head provided in one embodiment of the application;

FIG. 20 is a schematic front view of the structure of the transmitting head in FIG. 19;

FIG. 21 is a schematic cross-sectional structure diagram taken along the line C-C in FIG. 20;

FIG. 22 is a schematic diagram of the layout structure of the transmitting head provided in Embodiment 6 of the application;

FIG. 23 is a schematic diagram of the layout structure of the transmitting head provided in Embodiment 7 of the application.

Among them, the reference numbers in the drawings are mainly referred to:

100—Towel disinfecting and drying machine;

10—Main machine; 11—Casing; 110—Control panel; 111—Bottom plate; 112—Cover plate; 1121—Hook; 1122—Wire concealing groove; 113—Air outlet; 114—Air inlet; 115—Through hole; 1151—Heat dissipating hole; 1152—Fastener; 1153—Bump; 116—Venting hole; 117—Window; 118—Opening;

12—Heater; 121—Heating plate; 122—Heat dissipating fin;

13—Fan; 131—Wind hood; 132—Tubular wind wheel; 133—Motor;

14—Ultraviolet LED module; 141—Ultraviolet LED lamp bead; 142—Basal plate;

15—Wind guiding window; 151—Receiving groove;

161—Lamp socket; 162—Ultraviolet lamp tube; 163—Reflector; 1631—Supporting plate; 1632—Reflective side plate; 1633—Air flow hole; 164—Turntable; 1641—Rotating plate; 1642—Rotating shaft; 1643—Clamping socket; 165—Supporting seat; 166—Grille window;

18—Transmitting head; 181—Negative ion releasing brush; 182—Base; 183—Guide plate;

20—Hanger.

### DETAILED DESCRIPTION

In order to make the technical problems, technical solutions, and beneficial effects to be solved by this application clearer, the following further describes this application in detail with reference to the accompanying drawings and embodiments. It should be understood that the specific embodiments described here are only used to explain the application, but not to limit the application.

In the description of the present application, “a plurality of” means two or more than two, unless otherwise specifically defined. “Several” means one or more than one, unless otherwise specifically defined.

References described in the specification of this application to “one embodiment”, “some embodiments” or “embodiments” mean that one or more embodiments of the present application include a specific feature, structure, or characteristic described in conjunction with the embodiment. Therefore, “in one embodiment”, “in some embodiments”, “in some other embodiments”, “in yet other embodiments”, etc. in different places of this specification are not necessarily referred to the same embodiment, but mean “one or more, but not all embodiments”, unless otherwise specifically described. In addition, in one or more embodiments, specific features, structures, or characteristics may be combined in any suitable manner.

The English phrase corresponding to the English abbreviation used in this application is as follows:

LED, Light-emitting Diode.

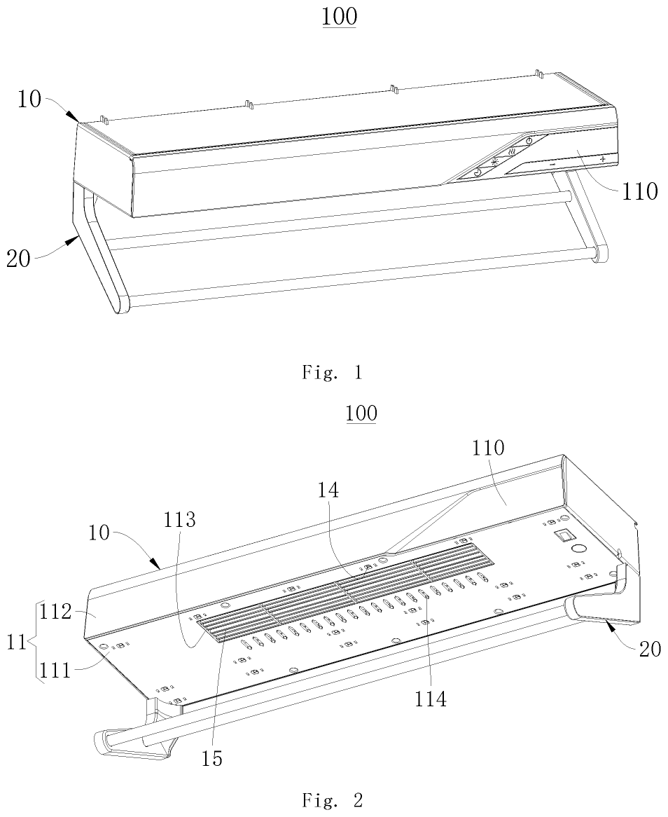

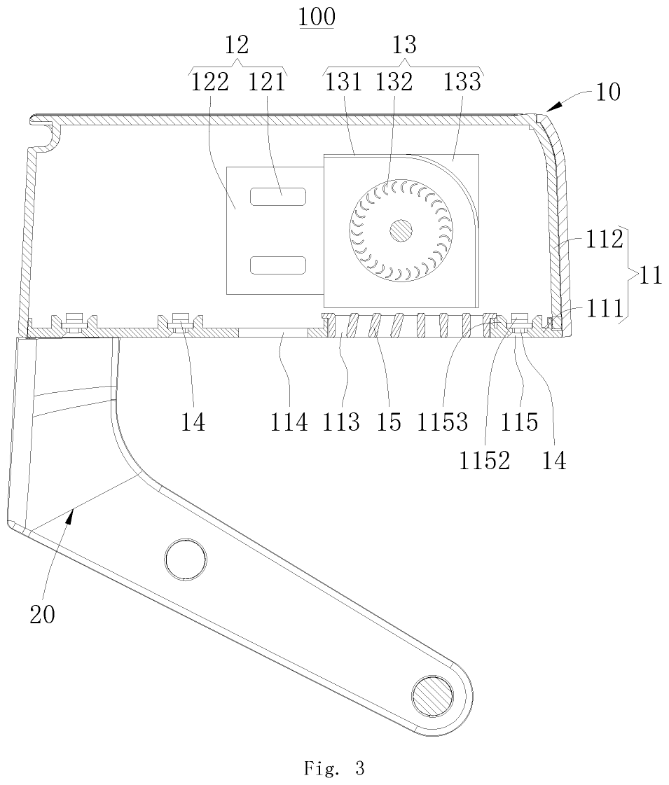

For the convenience of description, please refer to FIGS. 1 and 3, which defines in this application: when the main machine 11 is installed on a wall, the main machine 11, the casing 11 and the back side of the towel disinfecting and drying machine 100 are on the side of the main machine 11 close to the wall, and the main machine 11, the casing 11 and the front side of the towel disinfecting and drying machine 100 are on the side of the main machine 11 away from the wall.

Referring to FIGS. 1 to 3, the towel disinfecting and drying machine 100 provided in the present application will be described now. The towel disinfecting and drying machine 100 includes a main machine 11 and a hanger 20, wherein the hanger 20 is used to hang fabrics, that is, fabrics such as towels can be hung on the hanger 20, and the main machine 11 is installed above the hanger 20, so that the fabrics on the hanger 20 are heated, dried and disinfected by the main machine 11. The hanger 20 can be supported on the bottom of the casing 11 to integrate the hanger 20 and the main machine 11 together to facilitate installation and use. Of course, in some embodiments, the hanger 20 can also be separately provided to install the hanger 20 and the main machine 11 on the wall, respectively. In some other embodiments, the towel disinfecting and drying machine 100 can also only include the main machine 11, and the main machine 11 is installed above the existing hanger 20 in the room.

Referring to FIGS. 3 to 5, the main machine 11 includes a casing 11, an ultraviolet LED module 14, a heater 12 and a fan 13. The heater 12 and the fan 13 are installed in the casing 11, and protected by the casing 11. The bottom of the casing 11 is provided with an air inlet 114 and an air outlet 113, and the air outlet 113 is located at the outlet of the fan 13 for blowing out air flow. The heater 12 is arranged in the air flow path of the fan 13, and the heater 12 is used to heat the air, and the fan 13 blows the heated air out to dry the fabrics. The ultraviolet LED module 14 is installed on the bottom of the casing 11 to support the ultraviolet LED module 14 by the casing 11. The ultraviolet LED module 14 includes a basal plate 142 and an ultraviolet LED lamp bead 141. The basal plate 142 is installed on the bottom of the casing 11, and the ultraviolet LED lamp bead 141 is installed on the basal plate 142 to support and control the ultraviolet LED lamp bead 141 by the basal plate 142, so that the ultraviolet LED lamp bead 141 emits ultraviolet light to disinfect the fabrics. Since the size of the ultraviolet LED module 14 is relatively small, the occupied space in the casing 11 is small, and the casing 11 can be made smaller, and the position layout of the ultraviolet LED module 14 can be facilitated, and the area covered by the ultraviolet light emitted by ultraviolet LED module 14 can be easily controlled. Using the ultraviolet LED module 14 which is energy-efficient and is easy to install and fix, will simplify the structure of the main machine 11, facilitate assembly, the processing and production of the main machine 11.

Compared with the prior art, the towel disinfecting and drying machine 100 provided by the present application is provided with a fan 13 and a heater 12 in the casing 11 of the main machine 11, and the heat created by the heater 12 is blown out by the fan 13 for drying; the ultraviolet LED module 14 is installed on the bottom of the casing 11 to emit ultraviolet light by the ultraviolet LED lamp beads 141 for disinfecting, which can simplify the structure of the main machine 11 and also is energy-efficient, has long service life; because of the small size of the ultraviolet LED module 14, easy layout, and small occupied space, the main machine 11 can be made smaller, which is convenient for users to install and use.

In one embodiment, referring to FIGS. 1 to 3, the casing 11 includes a bottom plate and a cover plate 112 covering the bottom plate, so that the bottom plate constitutes the bottom of the casing 11, and the air outlet 113 is provided on the bottom plate. The casing 11 is easy to process, and also convenient to install and fix the heater 12, the fan 13 and the ultraviolet LED module 14. Of course, in other embodiments, the casing 11 can also be formed by a combination of multiple plates.

In one embodiment, referring to FIGS. 1 and 2, a control panel 110 is provided on the front side of the casing 11, and the control panel 110 is inclined in the direction toward the rear side of the casing 11 from top to bottom to facilitate user operations and determination of the position of the control panel 110 by users. In one embodiment, the control panel 110 is provided on the cover plate 112 to facilitate processing and manufacturing.

In one embodiment, there are a plurality of ultraviolet LED modules 14 to increase the coverage area of ultraviolet light and facilitate the installation and layout of each ultraviolet LED module 14. In some other embodiments, a single ultraviolet LED module 14 can also be provided. Specifically, the ultraviolet LED module 14 may be provided at a corresponding position according to user needs.

In one embodiment, referring to FIG. 2, the ultraviolet light emitted by the ultraviolet LED module 14 covers the hanger 20, so that the fabrics on the hanger 20 can be disinfected well.

In one embodiment, a plurality of ultraviolet LED modules 14 may be arranged on the bottom of the casing 11 to increase the coverage area of the ultraviolet light, so that the emitted ultraviolet light better covers the hanger 20 and facilitates the installation and layout of each of the ultraviolet LED module 14. In some other embodiments, a single ultraviolet LED module 14 can also be provided.

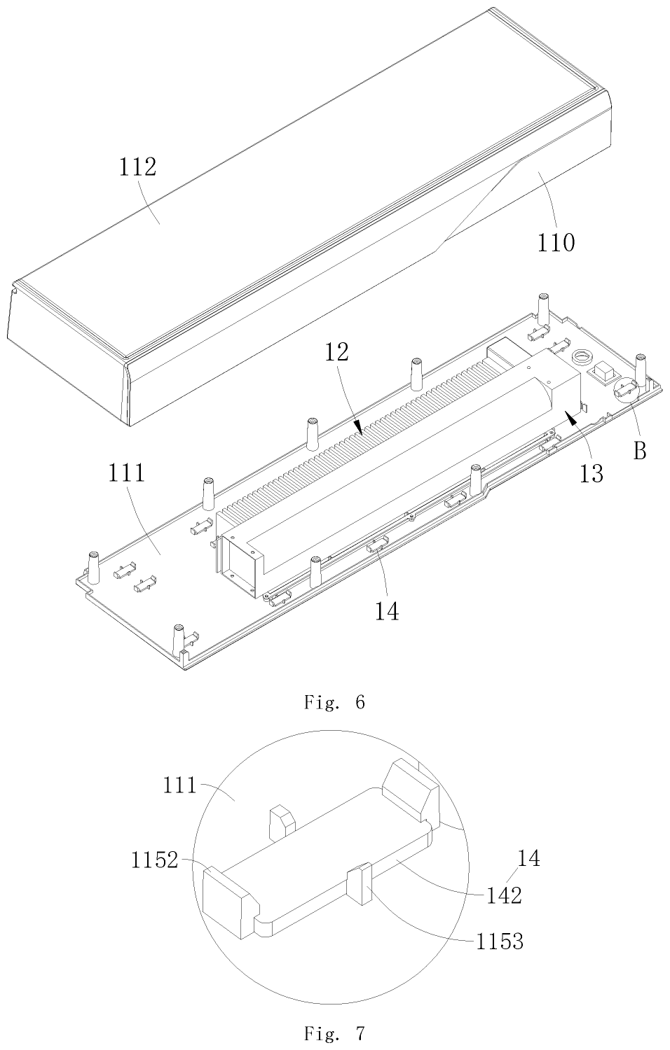

In one embodiment, referring to FIGS. 3 and 6, the ultraviolet LED module 14 is installed inside the casing 11 and protected by the casing 11, and the bottom of the casing 11 is provided with a through hole 115 which is located at a position corresponding to the ultraviolet LED module 14 to expose the corresponding ultraviolet LED lamp bead 141, and the ultraviolet light emitted by the corresponding ultraviolet LED lamp bead 141 can irradiate. In some other embodiments, the ultraviolet LED module 14 can also be installed outside the organic casing 11, so that the ultraviolet LED module 14 can be easily installed, and selected by the used.

In one embodiment, referring to FIGS. 3, 5 and 7, the cross-sectional area of the through hole 115 on the casing 11 is gradually expanded from the inner side of the casing 11 to the outer side of the casing 11, so that the through hole 115 can limit the light-emitting angle of the ultraviolet LED lamp bead 141, and the ultraviolet light emitted by the ultraviolet LED lamp bead 141 can cover a larger area. In some embodiments, a lens can also be covered on the ultraviolet LED lamp bead 141 to adjust the light-emitting angle of the ultraviolet LED lamp bead 141.

In one embodiment, the inner surface of the through hole 115 on the casing 11 is provided with a reflective layer (not shown), so that the through hole 115 forms a reflective cup, which improves the utilization rate of light emitted by the ultraviolet LED lamp beads 141. In some other embodiments, a reflective cup can also be separately provided to reflect the light emitted by the ultraviolet LED lamp beads 141.

In one embodiment, referring to FIGS. 3, 5 and 7, a fastener 1152 is provided on the bottom of the casing 11, so that the basal plate 142 can be fixed by the fastener 1152 to facilitate the installation and fixation of the ultraviolet LED module 14. When the ultraviolet LED module 14 is installed inside the casing 11, the fastener 1152 is also arranged inside the casing 11. When the ultraviolet LED module 14 is installed outside the casing 11, the fastener 1152 is also arranged outside the casing 11.

In one embodiment, the fasteners 1152 are arranged in pairs, and the pair of fasteners 1152 is respectively clamped to the two ends of the corresponding basal plate 142. The bottom of the casing 11 is also provided with bumps 1153 respectively positioned on both sides of the corresponding basal plate 142, thereby when the ultraviolet LED module 14 is installed, the basal plate 142 can be placed between the two corresponding bumps 1153, and a pair of fasteners 1152 can be clamped at both ends of the basal plate 142 to fix the basal plate 142, which is convenient for installation and fixation and can position accurately and also convenient to process the fastener 1152 and the bump 1153. In some other embodiments, the fasteners 1152 can also be used to clamp the four sides of the basal plate 142 to fix the basal plate 142. In some other embodiments, screws can also be used to fix the basal plate 142.

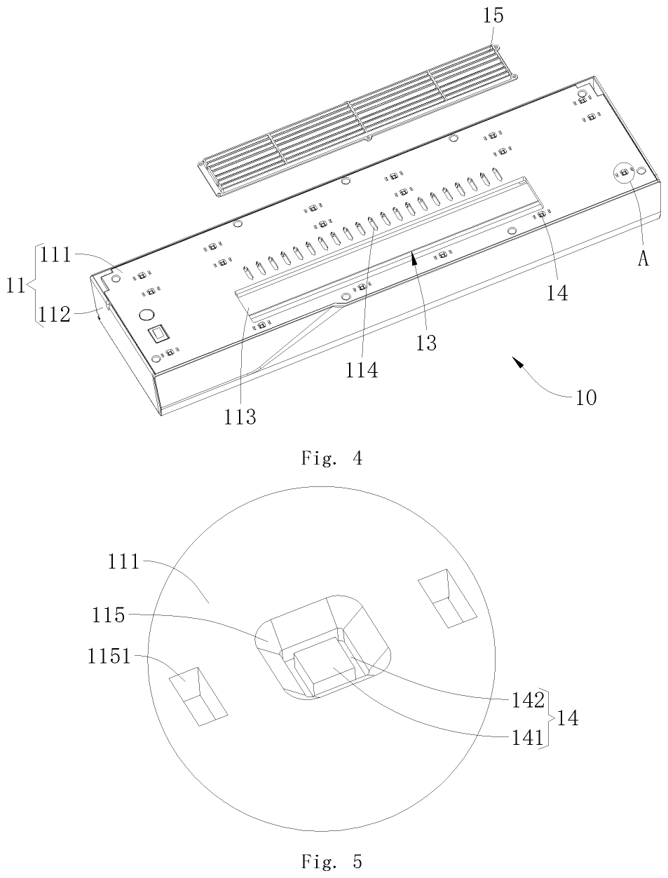

In one embodiment, referring to FIGS. 4 and 5, when the ultraviolet LED module 14 is installed inside the casing 11, the bottom of the casing 11 is provided with heat dissipating holes 1151 at the positions corresponding to the basal plates 142, so that heat of the basal plate 142 can be dissipated through the heat dissipating holes 1151.

In one embodiment, the corresponding position of each fastener 1152 is provided with a heat dissipating hole 1151, which can facilitate the integral injection molding of the fastener 1152 and the casing 11, and it is convenient for processing and manufacturing.

In one embodiment, referring to FIGS. 3 and 6, the fan 13 includes a wind hood 131, a tubular wind wheel 132, and a motor 133. The motor 133 is connected to the tubular wind wheel 132 to drive the tubular wind wheel 132 to rotate by the motor 133. The tubular wind wheel 132 is installed in the wind hood 131, and the air flow is guided by the wind hood 131. By using the tubular wind wheel 132, the volume can be made smaller, and the air volume can be large, and also the main machine 11 can be made smaller. Of course, in other embodiments, other fan 13 structures can also be used.

In one embodiment, referring to FIGS. 3 and 6, the heater 12 may be arranged at the inlet of the fan 13, and the air is heated by the heater 12 and then sucked and accelerated by the fan 13 before being blown out. Since the air flow velocity at the inlet of the fan 13 is relatively small, the gas can be fully heated by the heater 12 first, and then enter the fan 13, so that the heat utilization rate is high, and the power of the corresponding heater 12 can be made low. In other embodiments, the heater 12 can also be arranged at the outlet of the fan 13, and the air flow blown by the fan 13 is heated by the heater 12 before flowing out.

In one embodiment, the heater 12 is located at the inlet of the wind hood 131, and the air inlet 114 is located at a position corresponding to the heater 12, and the air is heated by the heater 12, and then sucked and accelerated by the fan 13 before being blown out.

In one embodiment, referring to FIGS. 3, 4, and 6, the heater 12 is provided with an ultraviolet LED module 14 on the side of the heater 12 away from the wind hood 131 to guarantee the area covered by the ultraviolet light, so as to better disinfect the fabrics under the main machine 11.

In one embodiment, referring to FIGS. 3, 4, and 6, an ultraviolet LED module 14 is provided on the side of the heater 12 away from the wind hood 131, and an ultraviolet LED module 14 is provided on the side of the wind hood 131 away from the heater 12. That is to say, the front and rear sides of the heater 12 and the wind hood 131 as a whole are provided with ultraviolet LED modules 14 respectively, which can reduce the blind area uncovered by ultraviolet light, due to the bottom space of casing 11 occupied by the heater 12 and the wind hood 131; In turn, it is ensured that the ultraviolet light emitted by the ultraviolet LED module 14 better covers the area under the main machine 11.

In one embodiment, at least two rows of ultraviolet LED modules 14 are provided near the rear side of the casing 11, and a row of ultraviolet LED modules 14 are provided near the front side of the casing 11. Since the rear side of the casing 11 is often close to the wall during use, it is difficult to be irradiated by external light, so at least two rows of ultraviolet LED modules 14 are arranged near the rear side of the casing 11 to increase the intensity of ultraviolet light, so that the fabrics near the wall can be better disinfected.

In one embodiment, the heater 12 includes a heating plate 121 and a plurality of heat dissipating fins 122, and the plurality of heat dissipating fins 122 are arranged on the heating plate 121 to heat the air more efficiently and quickly. The use of the heating plate 121 has high safety. Of course, in some other embodiments, the heater 12 can also use heating wires, heating tubes, etc.

In one embodiment, the main machine 11 further includes a wind guiding window 15 installed in the air outlet 113 to guide the air flow to diffuse, that is, when passing through the wind guiding window 15, the air flow blown by the fan 13 is diffused by the wind guiding window 15 to better heat and dry the fabrics.

In one embodiment, shutters may be used as the wind guiding window 15. In some other embodiments, a guide plate can also be used as the wind guiding window 15 to guide the air flow to diffuse.

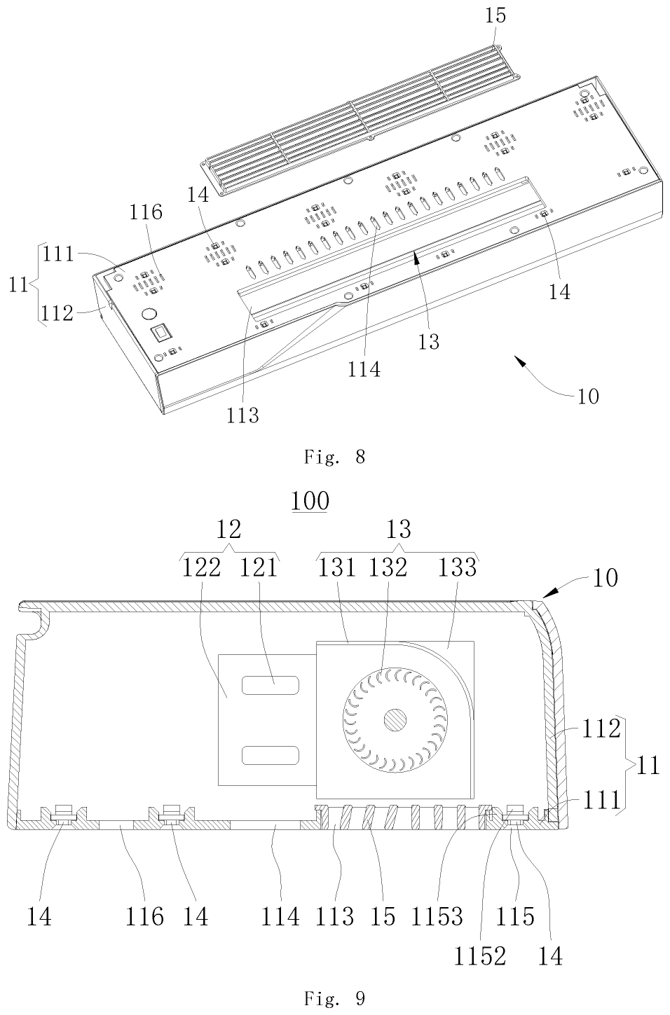

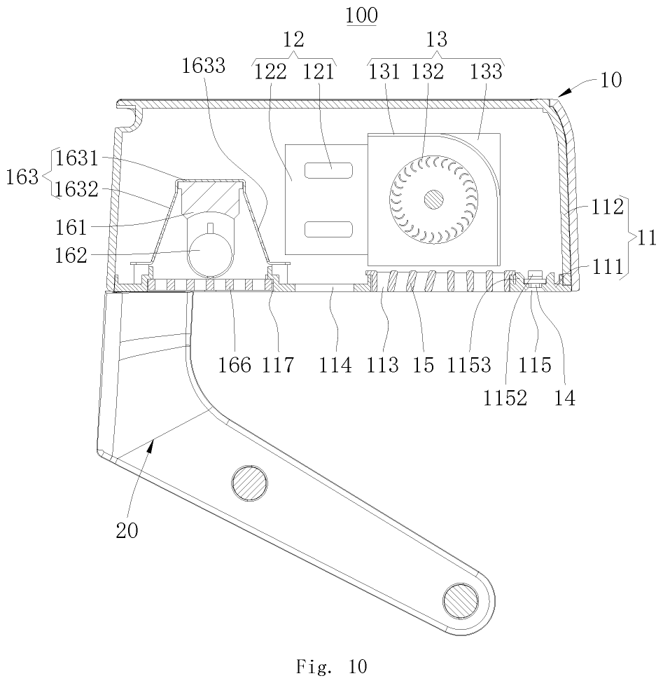

In one embodiment, referring to FIGS. 8 and 9, the bottom of the casing 11 is provided with a plurality of venting holes 116, and the venting holes 116 are arranged adjacent to the ultraviolet LED module 14 on the side of the heater 12 away from the wind hood 131, that is, the bottom of the casing 11 is provided with a plurality of venting holes 116 at the corresponding positions of the ultraviolet LED module 14 adjacent to the side of the heater 12 away from the wind hood 131. Since the heater 12 is located at the inlet of the wind hood 131, a venting hole 116 is provided at the corresponding position of the ultraviolet LED module 14 on the side of the heater 12 away from the wind hood 131, so that air can be sucked through the venting hole 116 to take away the heat generated near the LED module 14 and enters the heater 12 and then the wind hood 131, so that the corresponding ultraviolet LED module 14 can be dissipated, and this part of the heat can be used so that energy utilization is improved and energy consumption is reduced. The heat generated by the ultraviolet LED module 14 near the outlet of the wind hood 131 will also be taken out under the siphon effect of the wind from the wind hood 131. The ultraviolet LED module 14 can also be dissipated and the heat generated by the ultraviolet LED module 14 can be used so that the energy consumption is reduced.

In one embodiment, referring to FIGS. 8 and 9, the outlet of the wind hood 131 is located at a corresponding position near the front side of the casing 11, and the corresponding venting hole 116 is located at the position corresponding to the ultraviolet LED module 14 adjacent to the bottom of the casing 11 near the rear side of the casing 11. In some other embodiments, when the outlet of the wind hood 131 is located near the rear side of the casing 11, the corresponding venting hole 116 is located at the position corresponding to the ultraviolet LED module 14 adjacent to the bottom of the casing 11 near the front side of the casing 11.

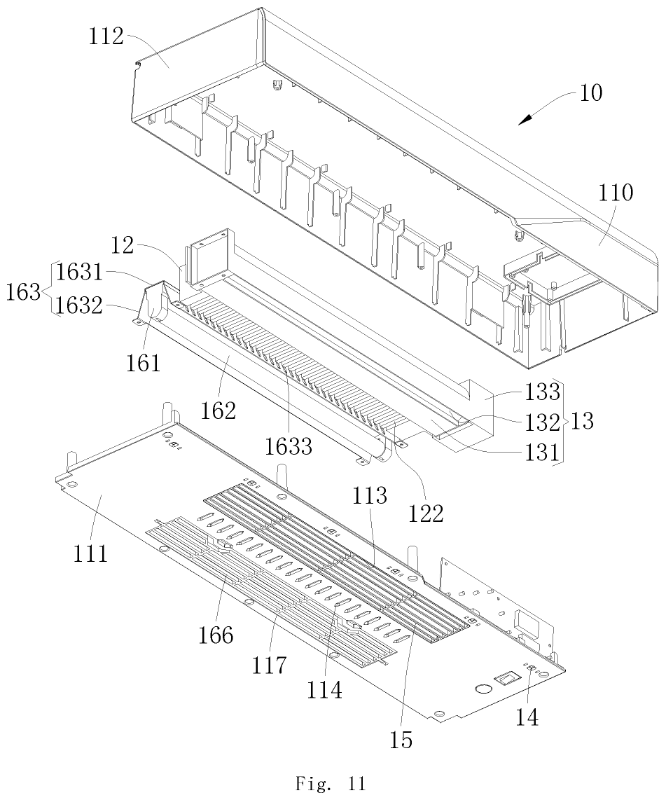

In one embodiment, referring to FIGS. 10 to 11, the main machine 11 further includes a lamp socket 161 and an ultraviolet lamp tube 162. The lamp socket 161 is installed in the casing 11, and protected by the casing 11. The ultraviolet lamp tube 162 is installed on the lamp socket 161, and the lamp socket 161 supports the ultraviolet lamp tube 162 and supplies power to the ultraviolet lamp tube 162, so that the ultraviolet lamp tube 162 emits ultraviolet light to disinfect the fabrics. A window 117 is provided on the bottom of the casing 11, and the window 117 is located at a position corresponding to the ultraviolet lamp tube 162 to expose the ultraviolet lamp tube 162, so that the ultraviolet light emitted by the ultraviolet lamp tube 162 can irradiate. The ultraviolet lamp tube 162 is provided to reduce the cost, and can cooperate with the ultraviolet LED module 14 to increase the coverage area to disinfect.

In one embodiment, referring to FIGS. 10 and 11, the main machine 11 further includes a reflector 163 installed in the casing 11, and protected by the casing 11. The reflector 163 is covered on the lamp socket 161, and when the ultraviolet lamp tube 162 is installed on the lamp socket 161, the reflector 163 can be covered on the ultraviolet lamp 162 so as to reflect the ultraviolet light emitted by the ultraviolet lamp 162 to a designated direction, improve the utilization of ultraviolet light and make it more energy-saving, and prevent the ultraviolet light emitted by the ultraviolet lamp tube 162 from irradiating other parts in the casing 11 and aging the corresponding parts.

In one embodiment, referring to FIGS. 10 and 11, the ultraviolet lamp tube 162 and the ultraviolet LED module 14 are located on both sides of the fan 13 and the heater 12 respectively, that is, the fan 13 and the heater 12 are located between the ultraviolet lamp tube 162 and the ultraviolet LED modules 14. In this embodiment, an ultraviolet lamp tube 162 is provided on the side of the heater 12 away from the fan 13, and an ultraviolet LED module 14 is provided on the side of the fan 13 away from the heater 12. In other embodiments, the side of the heater 12 away from the fan 13 is provided with an ultraviolet LED module 14, and the side of the fan 13 away from the heater 12 is provided with an ultraviolet tube 162. In this way, the ultraviolet lamp tube 162 and the fan 13 will occupy a larger area in the casing 11, which will make the main machine 11 on the side of the fan 13 away from the ultraviolet lamp tube 162 difficult to be covered by ultraviolet light, while the side of the fan 13 away from the ultraviolet lamp tube 162 is provided with the ultraviolet LED module 14, which can increase the coverage area of ultraviolet light and enhance the disinfection effect of the main machine 11.

In one embodiment, referring to FIGS. 10 and 11, the reflector 163 is located on the air intake path of the fan 13, and the reflector 163 is provided with air flow holes 1633. Air enters the reflector 163 through the window 117, and then the fan 13 through the air flow hole 1633, so that the lamp socket 161 and the ultraviolet lamp tube 162 in the reflector 163 can be dissipated, to increase the service life of the ultraviolet lamp tube 162 and the lamp socket 161. Moreover, after the air enters the reflector 163, it is heated by the ultraviolet lamp tube 162, so that the heat generated by the ultraviolet lamp tube 162 can be used, further improving the energy utilization rate and reducing the power consumption. The reflector 163 is provided to improve the utilization rate of the light emitted by the ultraviolet lamp tube 162. The reflector 163 is arranged on the air intake path of the fan 13, and an air flow hole 1633 is provided on the reflector 163, so that the air flow will pass through the reflector 163 and enter the fan 13, then is blown out, to dissipate and cool the ultraviolet lamp tube 162, and increase the service life of the ultraviolet lamp tube 162, and the heat generated by the ultraviolet lamp tube 162 can be used to heat the air flow at the inlet of the fan 13 to increase energy utilization rate and reduce energy consumption.

In one embodiment, the window 117 is located at a position corresponding to the bottom rear side of the casing 11, so that when in use, the window 117 is closer to the wall, so that the ultraviolet lamp tube 162 is also closer to the wall to better limit the range of ultraviolet light and enhance safety.

In one embodiment, referring to FIGS. 10 and 11, the reflector 163 has an air flow hole 1633 on the side close to the fan 13, so that after air enters the reflector 163, it can easily enter the fan 13 through the air flow hole 1633, reducing air resistance. Of course, in other embodiments, the air flow holes 1633 may be provided on both sides of the reflector 163 respectively. In some other embodiments, an air flow hole 1633 may be provided on the side of the reflector 163 away from the fan 13.

In one embodiment, the ultraviolet lamp tube 162 can be detachably installed on the lamp socket 161, so it is convenient to replace the ultraviolet lamp tube 162.

In one embodiment, referring to FIGS. 10 and 11, the reflector 163 includes two reflective side plates 1632 and a supporting plate 1631, and the air flow holes 1633 are provided on the reflective side plates 1632. The supporting plate 1631 is connected to the upper side of the two reflective side plates 1632, that is, the supporting plate 1631 is connected to the side of the two reflective side plates 1632 away from the window 117. When in use, the two reflective side plates 1632 are located on both sides of the lamp socket 161. Furthermore, when the ultraviolet lamp tube 162 is installed on the lamp socket 161, the two reflective side plates 1632 are located on both sides of the ultraviolet lamp tube 162 to reflect light. The distance between the two reflective side plates 1632 is gradually expanded from the supporting plate 1631 to the direction away from the supporting plate 1631, so that on the one hand, the light emitted by the ultraviolet lamp tube 162 can be reflected, and the reflected light can also be guaranteed to cover a larger area.

In one embodiment, the reflector 163 may be stamped from a metal plate to facilitate processing and manufacturing. In addition, the reflector 163 can also be used to dissipate the ultraviolet lamp tube 162. Of course, in some embodiments, the reflector 163 can also be a plastic cover, and a reflective coating is provided on the inner surface of the plastic cover.

In one embodiment, the lower end of the reflective side plate 1632 can be fixed to the bottom of the casing 11, that is, the end of the reflective side plate 1632 away from the supporting plate 1631 is fixed to the bottom of the casing 11 to install the reflector 163 on the casing 11.

In one embodiment, the supporting plate 1631 of the reflector 163 can be fixedly connected to the lamp socket 161, and the lamp socket 161 is fixed in the casing 11 to support the reflector 163 by the lamp socket 161.

In some other embodiments, the lower end of the reflective side plate 1632 can be fixed to the bottom of the casing 11, and the supporting plate 1631 of the reflector 163 can be fixedly connected to the lamp socket 161 to guarantee the installation stability of the reflector 163.

In one embodiment, the main machine 11 further includes a grille window 166. The grille window 166 is detachably installed on the bottom of the casing 11 and the grille window 166 is covered on the window 117 to protect the ultraviolet lamp tube 162 in the reflector 163, and can transmit light and ventilate through the grill window 166.

In one embodiment, the grille window 166 may use a light-transmitting material, so that the light emitted by the ultraviolet lamp tube 162 can be emitted through the grille window 166 to improve the utilization rate of light.

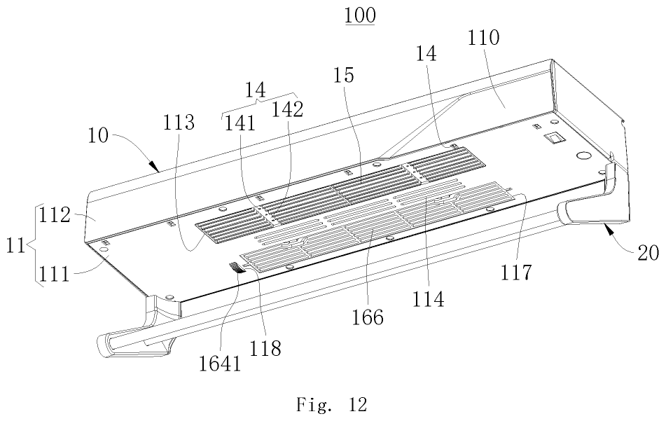

In one embodiment, referring to FIGS. 12 to 14, an ultraviolet LED module 14 is provided at the position corresponding to the air outlet 113. The ultraviolet LED module 14 is arranged at the position corresponding to the air outlet 113, and the air flow blown by the fan 13 passes through the air outlet 113, which can take away the heat generated by the ultraviolet LED module 14, so as to dissipate the ultraviolet LED module 14 and guarantee the service life of the ultraviolet LED module 14; and the air flow generated by the fan 13 will be blown to the fabric under the main machine 11, so that the heat generated by the ultraviolet LED module 14 can be brought to the fabric under the main machine 11 to utilize the heat generated by the ultraviolet LED module 14 and the heat generated by the heater 12 to bake the fabrics, which improves the energy utilization rate. In addition, the ultraviolet LED module 14 is arranged at the position corresponding to the air outlet 113, and the ultraviolet light generated by the ultraviolet LED module 14 can better cover the corresponding area under the air outlet 113, so that the fabric under the air outlet 113 can be dried and disinfected at the same time.

In one embodiment, referring to FIGS. 12 to 14, the basal plate 142 of the ultraviolet LED module 14 at the air outlet 113 is in an elongated form, and the basal plate 142 is supported in the air outlet 113 to facilitate supporting at the air outlet 113, and the area occupied by the basal plate 142 can be reduced to facilitate the air flow to dissipate heat from the basal plate 142, and the resistance of the basal plate 142 to the air flow can also be reduced, so that the air flow can heat the fabric under the main machine 11. Of course, in some other embodiments, the basal plate 142 can also be arranged in other shapes, such as a circle, a square, and so on. In some other embodiments, the basal plate 142 can also be arranged in a ring or frame shape to form a hole structure in the basal plate 142, or holes on the basal plate 142 can be directly formed to allow air flow to pass.

In one embodiment, referring to FIGS. 12 to 14, there are a plurality of ultraviolet LED modules 14, so that the power of a single ultraviolet LED module 14 can be made relatively small, which can reduce the heat generated by ultraviolet LED modules 14 and is convenient for each ultraviolet LED module 14 to dissipate heat, and ensure the service life of each ultraviolet LED module 14, and facilitate the position layout of each ultraviolet LED module 14, increase the area covered by the ultraviolet light. Of course, in some embodiments, there can also be a single ultraviolet LED module 14.

In one embodiment, referring to FIGS. 12 to 14, the basal plate 142 is in an elongated form, and when there are a plurality of ultraviolet LED modules 14, the basal plates 142 of the a plurality of ultraviolet LED modules 14 are arranged at intervals along the length direction of the air outlet 113, that is, the length direction of each basal plate 142 is perpendicular or slightly inclined to the length direction of the air outlet 113, so that the length of each basal plate 142 can be made shorter to guarantee the strength of each ultraviolet LED module 14 and it is also convenient to support at the air outlet 113. In other embodiments, when there are a plurality of ultraviolet LED modules 14, the basal plates 142 of the a plurality of ultraviolet LED modules 14 are arranged at intervals along the width direction of the air outlet 113, that is, the length direction of each basal plate 142 is perpendicular or slightly inclined to the width direction of the air outlet 113, so that the basal plate 142 can be made longer and the number of ultraviolet LED modules 14 can be reduced.

In the above embodiment, a plurality of ultraviolet LED lamp beads 141 are provided on each basal plate 142, so that the power of each ultraviolet LED lamp bead 141 can be made smaller, which can reduce the heat generated by each ultraviolet LED lamp bead 141 and facilitate the dissipation of ultraviolet LED lamp beads 141, guarantee the service life of each ultraviolet LED lamp beads 141, and facilitate the position layout of the ultraviolet LED lamp beads 141, increase the area covered by ultraviolet light. Of course, in some embodiments, there can also be a single ultraviolet LED lamp bead 141 on the basal plate 142.

In one embodiment, the wind guiding window 15 is provided with a receiving groove 151, and the ultraviolet LED module 14 is placed in the receiving groove 151, that is, the wind guiding window 15 is provided with a receiving groove 151 at a position corresponding to the ultraviolet LED module 14, to support the ultraviolet LED module 14 and reduce the volume of the main machine 11. In some embodiments, the ultraviolet LED module 14 can also be arranged under the wind guiding window 15. Of course, in other embodiments, the ultraviolet LED module 14 can also be arranged above the wind guiding window 15.

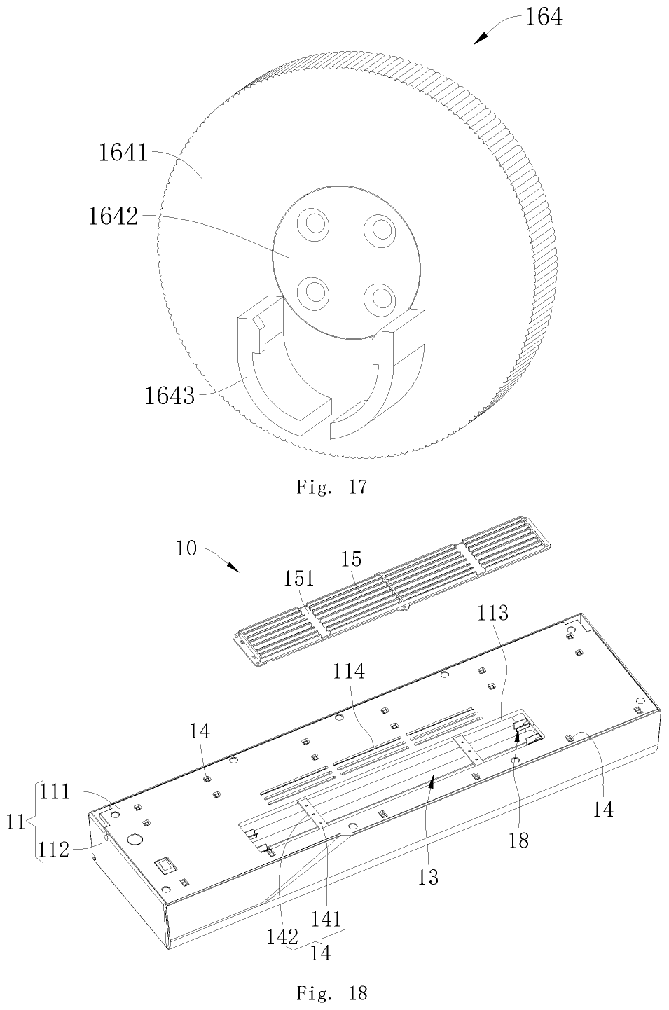

In one embodiment, referring to FIG. 17, a hook 1121 is provided on the rear side of the casing 11, so that the casing 11 can be mounted on the wall by hooking, which is convenient for installation and fixing. Of course, in some other embodiments, screws can also be used to install the casing 11 on the wall.

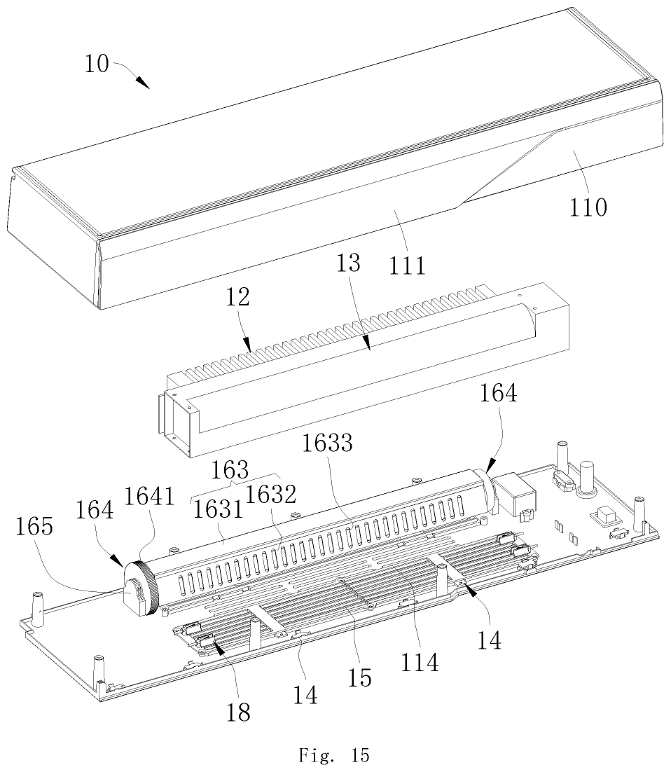

In one embodiment, referring to FIGS. 15 to 17, a wire concealing groove 1122 is provided on the rear side of the casing 11 so that the power supply can be placed in the wire concealing groove 1122.

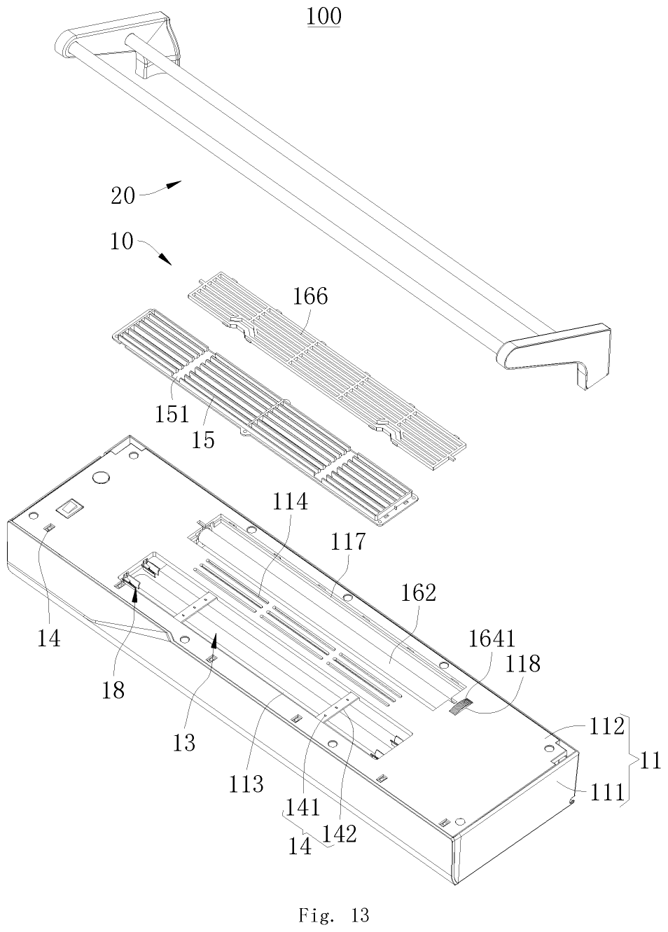

In one embodiment, referring to FIGS. 13-15, the side of the wind hood 131 away from the heater 12 is provided with an ultraviolet LED module 14 to increase the coverage area of ultraviolet light, and when the air flows out through the air outlet 113, a siphon effect is formed to suck away the hot air at the ultraviolet LED module 14 to form an air flow to dissipate heat from the ultraviolet LED module 14, and after the heat generated by the ultraviolet LED module 14 is sucked away, it can pass through the air outlet 113 and flow out for drying, which improves energy utilization.

In one embodiment, an ultraviolet LED module 14 is provided on the side of the heater 12 away from the wind hood 131, and the ultraviolet LED module 14 is supported on the bottom of the casing 11 to increase the area covered by the ultraviolet light, and thus the fabric under the main machine 11 is disinfected well.

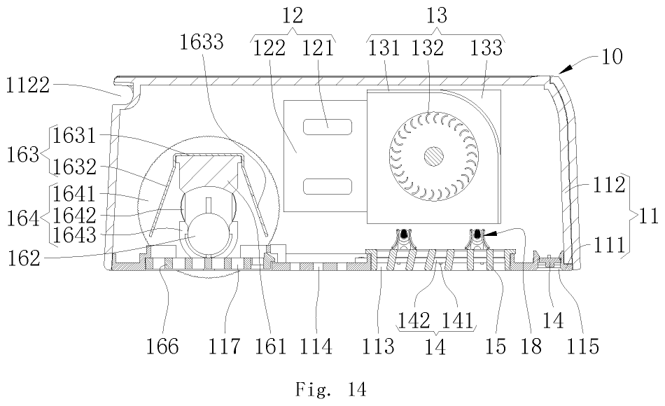

In one embodiment, referring to FIGS. 14 to 16, the reflector 163 can be fixedly connected to the lamp socket 161 to support the reflector 163 by the lamp socket 161. In other embodiments, the reflector 163 may be installed in the casing 11 and the reflector 163 is supported by the casing 11.

In one embodiment, the main machine 11 further includes two turntables 164 and two supporting seats 165. The two ends of the lamp socket 161 are respectively connected to the two turntables 164, and the reflector 163 is supported on the turntable 164. For example, the reflector 163 can be fixed on the lamp socket 161, the two ends of the reflector 163 can also be connected to two turntables 164, so that the lamp socket 161 and the reflector 163 are supported by the two turntables 164, and the two turntables 164 are respectively installed on the two supporting seats 165, two supporting seats 165 are respectively installed in the casing 11 to support the turntable 164 in the casing 11. A turntable 164 is provided to support the lamp socket 161 and the reflector 163. The turntable 164 can be rotated to drive the lamp socket 161, the ultraviolet lamp tube 162 and the reflector 163 to rotate, thereby adjusting the ultraviolet lamp tube 162 to emit ultraviolet light and the angle of the reflector 163 to reflect ultraviolet light. Then the coverage area of the ultraviolet lamp tube 162 is adjusted to disinfect the designated area.

In one embodiment, the side of the turntable 164 extends out of the casing 11, and the bottom of the casing 11 is provided with an opening 118 out of which the side of the turntable 164 extends, to facilitate the rotation of the turntable 164. In this embodiment, the diameter of a turntable 164 can be larger, so that the side of the turntable 164 extends out of the bottom of the casing 11 to facilitate the rotation of the turntable 164. In other embodiments, openings 118 may be provided on the casing 11 at the positions corresponding to two turntables 164, and the sides of the two turntables 164 respectively extend out of the bottom surface of the casing 11, so that the lamp socket 161 can be rotated more stably.

In one embodiment, referring to FIG. 17, the turntable 164 includes a rotating shaft 1642 and a rotating disk 1641. The rotating disk 1641 is rotatably mounted on the rotating shaft 1642. The rotating shaft 1642 is mounted on the supporting seat 165. The lamp socket 161 and the reflector 163 are on the rotating disk 1641, the rotating disk 1641 is supported by the rotating shaft 1642 to rotate, and when the rotating disk 1641 rotates, the lamp socket 161, the ultraviolet lamp tube 162 and the reflector 163 can be driven to rotate. In other embodiments, a disc can also be used as the turntable 164, and the disc is rotatably mounted on the supporting seat 165. In some other embodiments, the turntable 164 may be a rotating motor, and the lamp socket 161 and the reflector 163 are driven to rotate by the rotating motor to adjust the coverage area of the ultraviolet lamp tube 162.

In one embodiment, the rotating disc 1641 is further provided with a clamping socket 1643, which is used for clamping and fixing the lamp socket 161, that is, the lamp socket 161 can be clamped in the clamping socket 1643, and then the lamp socket 161 and the rotating disc 1641 is connected to facilitate assembly.

In one embodiment, the main machine 11 may include both the ultraviolet LED module 14 and the ultraviolet tube 162, so that the ultraviolet light can cover a larger area.

In one embodiment, referring to FIG. 18, the ultraviolet LED module 14 may be provided at the air outlet 113 at the same time, and the ultraviolet LED module 14 may be provided on the side of the heater 12 away from the wind hood 131.

In one embodiment, referring to FIGS. 14 to 16, the main machine 11 also includes a negative ion generator (not shown). The negative ion generator has a transmitting head 18, and the transmitting head 18 of the negative ion generator is arranged at a position corresponding to the air outlet 113. As a result, the air flow generated by the fan 13 passes through the transmitting head 18 of the negative ion generator, and then flows out of the air outlet 113 to form an air flow with negative ions, and then the fabrics are disinfected by the negative ions to improve the disinfection performance.

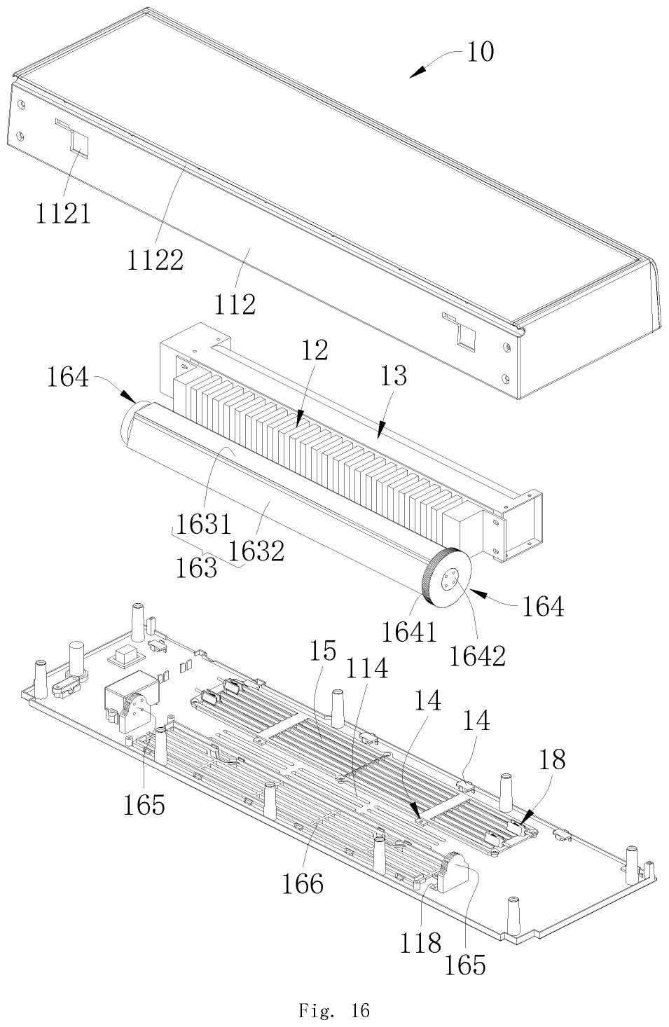

In one embodiment, referring to FIGS. 16, 19 and 20, the transmitting head 18 includes a negative ion releasing brush 181, a base 182 and two guide plates 183. The base 182 is installed on the casing 11, and the negative ion releasing brush 181 is installed on the base 182. The negative ion releasing brush 181 is supported by the support, and the negative ion releasing brush 181 is extended to the air outlet 113. The two guide plates 183 are respectively provided on the opposite sides of the negative ion releasing brush 181, the distance between the two guide plates 183 gradually expands downward from the middle of the guide plate 183, that is, the distance between the two guide plates 183 gradually expands in the direction from the middle of the guide plate 183 to the lower side of the guide plate 183, so that the space between the lower parts of the two guide plates 183 gradually increases, and the air flow to the lower part of the two guide plates 183 will slow down diffusion, and the negative ions in the air flow spread well to increase the area covered by negative ions.

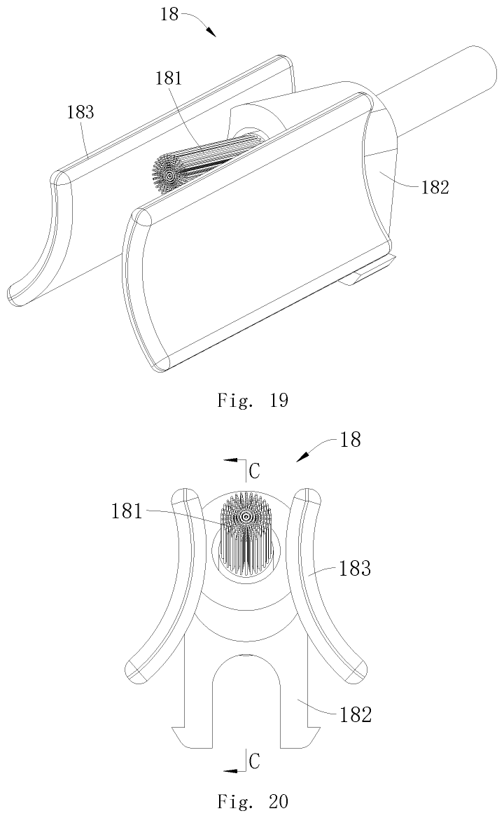

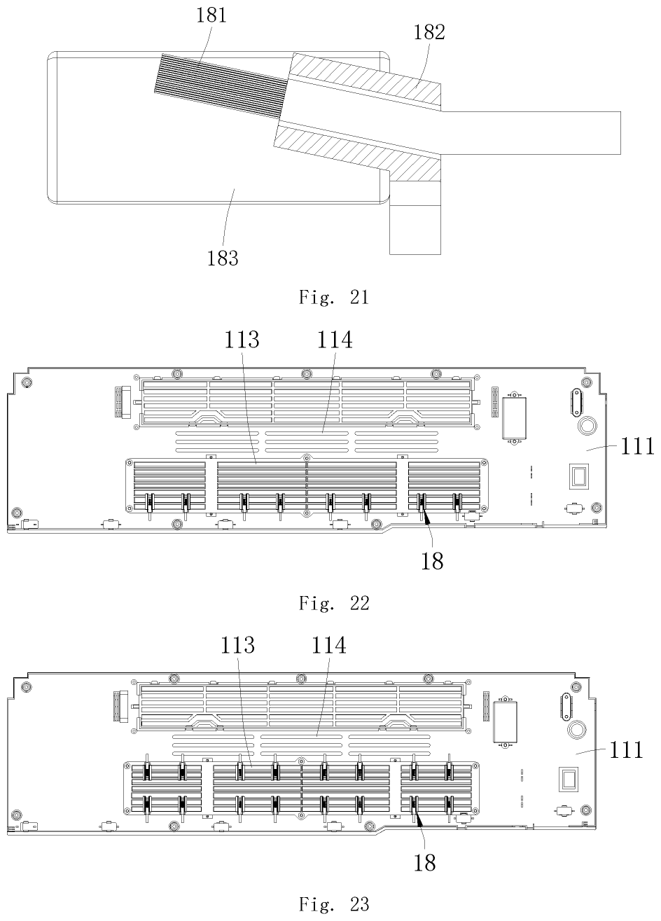

In one embodiment, referring to FIGS. 19 to 21, the guide plate 183 is fixedly connected to the base 182 to facilitate the installation and fixation of the guide plate 183 and further facilitate the installation and fixation of the transmitting head 18. Of course, in some embodiments, the guide plate 183 may be separately supported in the casing 11.

In one embodiment, the distance between the two guide plates 183 is gradually expanded upward from the middle of the guide plate 183, that is, the distance between the two guide plates 183 expanded in the direction from the middle of the guide plate 183 to the upper side of the guide plate 183, so that more air enters between the two guide plates 183, and contacts the negative ion releasing brush 181, thereby generating more negative ions.

In one embodiment, the distance between the upper sides of the two guide plates 183 is smaller than the distance between the lower sides of the two guide plates 183, so as to ensure that the air flow velocity from the lower sides of the two guide plates 183 is less than that out of the two outer guide plates 183. In this way, a certain siphon effect will be formed on the lower sides of the two guide plates 183, so as to better diffuse the negative ions in the air flow between the two guide plates 183, thereby increasing the coverage area of negative ions.

In one embodiment, the negative ion releasing brush 181 is located at a corresponding position above the middle of the guide plate 183, so that more air flow passes through the negative ion releasing brush 181, and then is guided and diffused by the guide plate 183.

In one embodiment, the negative ion releasing brush 181 extends obliquely upward, that is, the negative ion releasing brush 181 extends obliquely upward from the base 182 to the free end of the negative ion releasing brush 181. Since the free end of the negative ion releasing brush 181 is relatively more opened, the negative ion releasing brush 181 is extended obliquely upwards, which can reduce the resistance of the negative ion releasing brush 181 to the air flow, thereby facilitating the air flow through the negative ion releasing brush 181 to produce more negative ions.

In one embodiment, referring to FIG. 16, the two ends of the length direction of the air outlet 113 are respectively provided with a transmitting head 18, so that the negative ions generated by the transmitting head 18 can cover a larger area, to disinfect well the fabric under the main machine 11.

In one embodiment, along the width direction of the air outlet 113, the two ends of the length direction of the air outlet 113 are respectively provided with a plurality of transmitting heads 18, so that the negative ions can cover a larger area, to disinfect well the fabric under the main machine 11.

In one embodiment, referring to FIG. 22, the side of the length direction of the air outlet 113 is provided with a plurality of transmitting heads 18, so that the negative ions generated by the transmitting head 18 can cover a larger area, to disinfect well the fabric under the main machine 11.

In one embodiment, a plurality of transmitting heads 18 may be arranged on one side of the length direction of the air outlet 113, so that the negative ions generated by the transmitting head 18 can cover the entire length direction of the air outlet 113, to disinfect well the fabric under the main machine 11.

In one embodiment, referring to FIG. 23, a plurality of transmitting heads 18 are provided on both sides of the length direction of the air outlet 113, so that the negative ions generated by the transmitting head 18 can cover the entire air outlet 113, to disinfect well the fabric under the main machine 11.

The Towel disinfecting and drying machine 100 of the embodiments of the present application has small size, large air volume, low energy consumption, high energy utilization, long service life, simple structure, easy assembly, and a large coverage area of ultraviolet light.

The above are only optional embodiments of this application and are not intended to limit this application. Any modification, equivalent replacement and improvement made within the spirit and principle of this application shall be included in the protection scope of this application.

What is claimed is:

1. A towel disinfecting and drying machine, including a main machine, wherein the main machine includes a casing, an ultraviolet LED module installed on the bottom of the casing, a heater installed in the casing, and a fan installed in the casing, an air outlet is provided on the bottom of the casing corresponding to the outlet of the fan, the heater is provided in the air flow path of the fan, and an air inlet is arranged on the bottom of the casing, the ultraviolet LED module includes a basal plate installed on the bottom of the casing and ultraviolet LED lamp bead(s) installed on the basal plate.

2. The towel disinfecting and drying machine according to claim 1, wherein the bottom of the casing is provided with hooks for clamping and fixing the basal plate.

3. The towel disinfecting and drying machine according to claim 2, wherein the hooks are arranged in pairs, and a pair of the hooks is respectively clamped to the two ends of the corresponding basal plate, and the bottom of the casing is provided with bumps for positioning the two sides of the basal plate respectively.

4. The towel disinfecting and drying machine according to claim 1, wherein the ultraviolet LED module is installed inside the casing, and the bottom of the casing is provided with a through hole exposing each of the ultraviolet LED lamp beads, the cross-sectional area of the through hole is gradually expanded from the inner side of the casing to the outer side of the casing.

5. The towel disinfecting and drying machine according to claim 4, wherein the inner surface of the through hole is provided with a reflective layer.

6. The towel disinfecting and drying machine according to claim 1, wherein the ultraviolet LED module is provided at a corresponding position of the air outlet.

7. The towel disinfecting and drying machine according to claim 6, wherein the basal plate of the ultraviolet LED module at the air outlet is in an elongated form, and the basal plate is supported in the air outlet.

8. The towel disinfecting and drying machine according to claim 7, wherein there are a plurality of ultraviolet LED modules at the air outlet, and the basal plates of a plurality of ultraviolet LED modules are arranged at intervals along the length of the air outlet, or the basal plates of a plurality of the ultraviolet LED modules are arranged at intervals along the width direction of the air outlet.

9. The towel disinfecting and drying machine according to claim 7, wherein a plurality of the ultraviolet LED lamp beads are provided on each of the basal plates.

10. The towel disinfecting and drying machine according to claim 6, wherein the main machine further comprises a wind guiding window for guiding the diffusion of air flow, and the wind guiding window is installed in the air outlet.

11. The towel disinfecting and drying machine according to claim 10, wherein the wind guiding window is provided with a receiving groove for accommodating the ultraviolet LED module.

12. The towel disinfecting and drying machine according to claim 1, wherein the main machine further comprises a lamp socket installed in the casing and an ultraviolet lamp tube installed on the lamp socket, the bottom of the casing is provided with an opening window exposing the ultraviolet lamp tube.

13. The towel disinfecting and drying machine according to claim 12, wherein the main machine further comprises a reflector covering the ultraviolet lamp tube, the reflector is provided with air flow holes, and the reflector is located at the air intake path of the fan.

14. The towel disinfecting and drying machine according to claim 1, wherein the fan includes a wind hood installed in the casing, and a tubular wind wheel installed in the wind hood and a motor driving the rotation of the wind wheel, the heater is located at the inlet of the wind hood, and the air inlet is provided at a position corresponding to the heater.

15. The towel disinfecting and drying machine according to claim 14, wherein the ultraviolet LED module is provided on the side of the wind hood away from the heater.

16. The towel disinfecting and drying machine according to claim 14, wherein the ultraviolet LED module is provided on the side of the heater away from the wind hood.

17. The towel disinfecting and drying machine according to claim 16, wherein a plurality of venting holes are provided on the bottom of the casing, and the venting holes are arranged adjacent to the ultraviolet LED module on the side of the heater away from the wind hood.

18. The towel disinfecting and drying machine according to claim 1, wherein the towel disinfecting and drying machine further comprises a hanger for hanging fabrics, and the hanger is supported on the bottom of the casing.

19. The towel disinfecting and drying machine according to claim 1, wherein the front side of the casing is provided with a control panel, and the control panel is inclined in the direction toward the rear side of the casing from top to bottom.

20. The towel disinfecting and drying machine according to claim 1, wherein the main machine further comprises a negative ion generator, the negative ion generator has a transmitting head, and the transmitting head is arranged at the position corresponding to the air outlet.
